(19) United States
(12) Patent Application Publication
(10) Pub. No.: US 2026/0344980 A1
(43) Pub. Date: Nov. 5, 2026

(54) PARTIALLY REUSABLE MEDICAL DEVICES AND RELATED METHODS OF USE

(71) Applicants: Boston Scientific Scimed, Inc., Maple Grove, MN (US); Boston Scientific Medical Device Limited, Galway (IE)

(72) Inventors: Joseph Phillip ALBINA, Hudson, MA (US); Sean POWELL, Holden, MA (US); Shrikant Vasant RAUT, Pune (IN); Pooja Bhuvanesh KULKARNI, Pune (IN); Aditya DHANOTIYA, Pune (IN)

(73) Assignees: Boston Scientific Scimed, Inc., Maple Grove, MN (US); Boston Scientific Medical Device Limited, Galway (IE)

(21) Appl. No.: 19/664,156

(22) Filed: Apr. 30, 2026

Related U.S. Application Data
(60) Provisional application No. 63/798,895, filed on May 2, 2025.
(60) Provisional application No. 63/867,264, filed on Aug. 20, 2025.

(51) Int. Cl. A61B 1/00; A61B 1/015

(52) U.S. Cl. CPC A61B 1/00105; A61B 1/00066; A61B 1/00124; A61B 1/015

(57) ABSTRACT

A medical device comprises a first portion having a housing, an actuator, a motor configured to be activated by the actuator, a gear assembly coupled to the motor, and a second portion removably coupled to the first portion. The second portion comprises a spool coupled to a wire and a second gear coupled to the spool. A first gear of the gear assembly is configured to interact with the second gear to move the wire.

### CROSS-REFERENCE TO RELATED APPLICATIONS

This application claims the benefit of priority of U.S. Provisional Patent Application No. 63/798,895, filed May 2, 2025 and U.S. Provisional Patent Application No. 63/867,264, filed Aug. 20, 2025, each of which is incorporated herein by reference in its entirety.

### TECHNICAL FIELD

Various aspects of this disclosure relate generally to partially reusable medical devices and related methods of use. In particular, aspects of this disclosure relate to partially reusable medical devices including a reusable portion removably coupled to a disposable portion having a deflectable shaft.

### BACKGROUND

A scope device, such as a duodenoscope, an endoscope, or other scope device, may include a shaft coupled to a handle including actuators or controls. The shaft may include a working channel and a deflectable distal end. During a medical procedure, the shaft may be inserted into a body lumen of a subject (e.g., a patient). The actuators or controls on the handle may be used to deflect the distal end of the shaft to, e.g., change an orientation of a medical instrument extending through the working channel or a view provided by an imaging device at the distal end of the shaft. After the medical procedure, a reusable scope device may be reused after being disinfected. A disposable scope device may be disposed of in its entirety after the medical procedure. However, disposable and reusable scope devices may present challenges. For example, disposable scope devices may have high costs or increase medical waste. Reusable scope devices may be difficult to disinfect, which may increase a patient risk of infection.

### SUMMARY

Each of the aspects disclosed herein may include one or more of the features described in connection with any of the other disclosed aspects. For example, aspects of this disclosure relate to, among other things, handle devices and assemblies for medical devices. Aspects of this disclosure pertain to handles of medical devices, such as endoscopes or other scope devices.

In aspects, a medical device comprises a first portion having a housing, an actuator, a motor configured to be activated by the actuator, a gear assembly coupled to the motor, and a second portion removably coupled to the first portion. The second portion comprises a spool coupled to a wire and a second gear coupled to the spool. A first gear of the gear assembly is configured to interact with the second gear to move the wire.

Any of the embodiments disclosed herein may have any of the following features, alone or in any combination. The second gear of the spool extends proximally relative to a housing of the second portion. The motor is a first motor, the gear assembly is a first gear assembly, the spool is a first spool, and the wire is a first wire, and the first portion further has a second motor configured to be activated by the actuator and a second gear assembly coupled to the second motor. The second portion further has a second spool coupled to a second wire and a fourth gear coupled to the second spool. A third gear of the second gear assembly is configured to interact with the fourth gear to move the second wire. Each of the first motor and the second motor extend approximately perpendicularly to a central longitudinal axis of the medical device. The first motor is coupled to a first axle that rotates a fourth gear of the first gear assembly. The second motor is coupled to a second axle that rotates a fifth gear of the second gear assembly. The first axle and the second axle are approximately perpendicular to a central longitudinal axis of the medical device. The medical device further includes a shaft extending from the second portion. A distal end of the shaft includes an articulation portion. Movement of the first wire articulates the distal end of the shaft in a first direction and a second direction, opposite the first direction. Movement of the second wire articulates the distal end of the shaft in a third direction and a fourth direction opposite the third direction. The third direction and the fourth direction are perpendicular to the first direction and the second direction. The second gear assembly includes more gears than the first gear assembly. The first gear assembly includes three gears and the second gear assembly includes two gears. The first motor is proximal of the second motor. The first gear assembly is coupled to the first motor via a first axle. The second gear assembly is coupled to the second motor via a second axle. Each of the first axle and the second axle extends through a wall separating the first motor and the second motor from the first gear assembly and the second gear assembly, respectively. The first portion includes a first umbilicus. The first umbilicus includes at least one electrical wire or cable. The second portion includes a second umbilicus. The second umbilicus includes at least one fluidics tube. The second portion includes a first valve well configured to receive a valve body for controlling application of a fluid. The actuator is a first actuator, and the medical device further includes a shaft extending from a distal end of the second portion, a distal portion of the shaft including an articulation portion and an elevator. The medical device further includes a second actuator disposed on the first portion. The first actuator is configured to articulate the articulation portion of the shaft in at least one direction, and the second actuator is configured to actuate the elevator.

In another aspect, a medical device comprises a handle having a first portion including a motor and a first gear. The motor is configured to rotate the first gear. The handle has a second portion removably coupled to the first portion. The second portion includes a second gear configured to interact with the first gear of the first portion to rotate a spool and a wire coupled to the spool. The medical device further comprises a shaft extending from the second portion. A distal end of the shaft includes an articulation portion. The wire is coupled to the articulation portion such that, upon activation of the motor, the wire articulates the articulation portion in a first direction or a second direction, opposite the first direction.

Any of the embodiments disclosed herein may have any of the following features, alone or in any combination. The motor is a first motor, the gear is a first gear, and the wire is a first wire, and the first portion further comprises a second motor and a third gear. The second portion further comprises a fourth gear configured to interact with the third gear of the first portion to rotate a second spool and a second wire coupled to the second spool and to the articulation portion. Upon activation of the second motor, the second wire articulates the articulation portion of the shaft in a third direction or a fourth direction. The first gear is a gear of a first gear train. The third gear is a gear of a second gear train. The first gear train includes more gears than the second gear train.

In another aspect, a medical device comprises a first handle portion including an actuator and a first motor coupled to a first gear via a first axle. The first motor is configured to rotate the first gear. The first handle portion further includes a second motor coupled to a second gear via a second axle. The second motor is configured to rotate the second gear. The medical device also comprises a second handle portion. The second handle portion is removably coupled to the first handle portion. The second handle portion includes a first wire connected to a first spool. The first spool is configured to rotate to move the first wire proximally or distally. The second handle portion also includes a second wire connected to a second spool. The second spool is configured to rotate to move the second wire proximally or distally. Upon actuation of the actuator, the first gear rotates the first spool or the second gear rotates the second spool.

Any of the embodiments disclosed herein may have any of the following features, alone or in any combination. Actuation of the actuator is configured to simultaneously activate the first motor and the second motor in order to simultaneously rotate the first spool and the second spool.

The foregoing general description of the illustrative embodiments and the following detailed description thereof are merely exemplary aspects of the teachings of this disclosure and are not restrictive.

### BRIEF DESCRIPTION OF FIGURES

The accompanying drawings, which are incorporated in and constitute a part of this application, illustrate exemplary aspects of this disclosure and, together with the description, serve to explain the principles of this disclosure.

FIG. 1 depicts an exemplary medical device, according to aspects of this disclosure.

FIGS. 2A-2D depict a first portion of a handle of a medical device, according to aspects of this disclosure.

FIGS. 3A-3D depict a second portion of a handle of the medical device, according to aspects of this disclosure.

FIG. 4 depicts aspects of the first portion of the handle of FIGS. 2A-2D, according to aspects of this disclosure.

FIG. 5 depicts motors and gear assemblies of the first portion of the handle of FIGS. 2A-2D, according to aspects of this disclosure.

FIG. 6 depicts aspects of the first portion of the handle of FIGS. 2A-2D, according to aspects of this disclosure.

FIGS. 7A and 7B depict first and second gear trains, respectively, of the first portion of the handle of FIGS. 2A-2D, according to aspects of this disclosure.

FIGS. 8A and 8B depict an alternative exemplary medical device, according to aspects of this disclosure.

FIGS. 9A and 9B depict an exemplary graphical user interface, according to aspects of this disclosure.

### DETAILED DESCRIPTION

Particular aspects of the disclosure are described in greater detail below. The terms and definitions provided herein control, if in conflict with terms or definitions incorporated by reference. Wherever possible, the same or similar reference numbers will be used through the drawings to refer to the same or like parts. Where feasible, reference numbers ending in the same tens and ones digits refer to corresponding components.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “or” is used disjunctively, such that “at least one of A or B” includes, (A), (B), (A and A), (A and B), etc. The term “or” also includes “and/or.” Relative terms such as “about,” “substantially,” and “approximately,” etc., are used to indicate a possible variation of ±10 % of the stated numeric value or range.

This disclosure includes medical devices having a reusable portion (e.g., a first portion or a proximal portion) removably coupled to a disposable portion (e.g., a second portion or a distal portion). According to some aspects of this disclosure, features of the reusable portion and the disposable portion may allow the reusable portion to be, for example, easily connected to or disconnected from the disposable portion (e.g., physically or operably).

According to some aspects of this disclosure, the disposable portion of the medical device may include components that may be difficult to disinfect or clean. For example, the disposable portion of the medical device may include a deflectable shaft, one or more channels, one or more ports in communication with the channel(s), steering or other types of control wires, pulleys or cams, an elevator, or fluidic components (e.g., valves, tubing, or conduits for air, water, or suction).

According to some aspects of this disclosure, the reusable portion may include an actuator operable to activate motors of the reusable portion. The motors may be operatively coupled to gear assemblies within the reusable portion. The gear assemblies within the reusable portion may be configured to interact or engage with a pull wire mechanism (e.g., steering pulleys or cams) within the disposable portion to deflect a distal end of the shaft.

Although duodenoscopes are illustrated herein, it will be appreciated that the disposable portion described herein may be adapted to other scope devices, such as bronchoscopes, ureteroscopes, endoscopes, gastroscopes, endoscopic ultrasonography (“EUS”) scopes, colonoscopes, laparoscopes, cystoscopes, aspiration scopes, or any other type of scope device. It will be appreciated that although the reusable and disposable portions described herein may provide a four-way steering medical device, the reusable portion or the disposable portion may be adapted to provide a two-way steering medical device. In some aspects, the same reusable portion may be used with a variety of different types of disposable portions (e.g., the same reusable portion may be used with a side-viewing duodenoscope and a forward-facing gastroscope or any other type of scope).

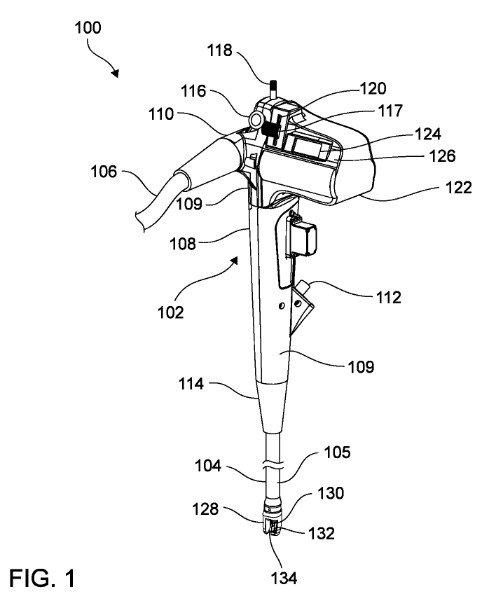

FIG. 1 depicts a medical device 100. Medical device 100 may have any of the properties of the medical devices or aspects thereof disclosed in U.S. Provisional Patent Application No. 63/798,639, entitled “Handle Systems and Assemblies for Medical Devices and Related Methods of Use,” filed on May 2, 2025, which is incorporated by reference in its entirety.

Medical device 100 may have a handle 102, a shaft 104 extending distally from handle 102, and an umbilicus 106. Umbilicus 106 may extend from a proximal portion of handle 102. Handle 102 may include a gripping portion 108 formed by one or more handle housing pieces 109. Handle housing pieces 109 may be formed from a rigid material, such as a medical-grade polymer, metal, or composite material, and may be configured to contain or provide structural support for the internal components of handle 102. Handle housing pieces 109 may include ergonomic contours (e.g., protrusions or indentations) or surface features (e.g., textured surfaces, roughened surfaces, tacky surfaces, etc.) designed to enhance grip comfort and control during extended medical procedures. In some aspects, handle housing pieces 109 may be formed as two or more separate pieces, such as a left housing portion and a right housing portion, that can be assembled to enclose the internal components of handle 102.

Handle 102 may include an umbilicus port 110 for connecting to umbilicus 106. Umbilicus 106 may provide connections for tubing, wiring, or cabling between handle 102 and one or more external fluid or electronic sources. For example, umbilicus 106 may be configured to connect handle 102 to fluidic sources such as fluidic pumps or suction sources. Additionally or alternatively, umbilicus 106 may connect handle 102 to one or more electronic sources including computers, controllers, or displays. Through umbilicus 106, fluids, data, or power may be transferred between handle 102 and these external sources, enabling various functionalities of medical device 100 during operation (e.g., insufflation, aspiration, fluid delivery, image capture, illumination, etc.).

Shaft 104 may extend distally from handle 102 and may be configured to be inserted into a body lumen of a subject. A strain relief 114 may be positioned at the junction of handle 102 and shaft 104. Strain relief 114 may be designed to reduce stress concentration and prevent kinking or damage to internal components at this transition point. At least a portion of shaft 104 may be flexible, semi-flexible, or rigid.

A distal end portion of shaft 104 may include an articulation portion 105 and a distal tip 128 that is fixed to a distal end of shaft 104. In some aspects, articulation portion 105 may include an articulation joint that is articulated or steered via controls (e.g., actuators) on handle 102, allowing a user to maneuver distal tip 128 within the body. For example, articulation portion 105 may be configured to bend in one or more directions relative to a longitudinal axis of shaft 104 (e.g., left, right, up, or down).

Distal tip 128 may include an imaging device 130 and an illumination device 132. Imaging device 13 may be capable of capturing still images or video footage. Such footage may be transmitted to handle 102 or an external display for viewing by the user (e.g., via umbilicus 106 or other means). Imaging device 130 may be side-facing (as shown) or forward-/distal-facing. Illumination device 132 may include one or more light sources, such as light-emitting diodes (LEDs) or optical fibers. Illumination device 132 may provide light to illuminate an area around distal tip 128, thereby enhancing visibility for the imaging device 130 and the user.

Distal tip 128 may further include an elevator 134 configured to change the angle or direction of instruments or tools that are passed through medical device 100. For example, an instrument or tool may be inserted through a port 112 of handle 102 that is in communication with a lumen or a working channel of shaft 104. The instrument or tool may be extended through the lumen or working channel and out of a distal opening of distal tip 128. In some aspects, elevator 134 may be configured to enable the user to adjust the position or orientation of instruments exiting the distal tip 128 during a procedure. Described in further detail below, a position of elevator 134 or a speed at which elevator 134 may be actuated may be controlled by one or more actuators on handle 102.

Handle 102 may include one or more actuators for controlling various functions of medical device 100. Such actuators may include a first actuator 116, a second actuator 117, or a third actuator 118. Although three actuators are shown, medical device 100 may include fewer actuators or additional actuators. Such actuators may be buttons, knobs, sliders, levers, joysticks, switches, etc. Each actuator may be configured to control one or more functions of medical device 100. While specific functions for each actuator are described below, it should be understood that the actuators may be interchangeable or multifunctional. For example, first actuator 116 may be configured to accomplish the same functions as second actuator 117 or third actuator 118. Similarly, second actuator 117 may be configured to perform the same functions as first actuator 116 or third actuator 118. Third actuator 118 may likewise be configured to perform the same functions as first actuator 116 or second actuator 117. Additional actuators may control the same functions as first actuator 116, second actuator 117, third actuator 118, or other functions.

In some aspects, first actuator 116 may be a joystick configured to control a position or configuration of articulation portion 105, and, therefore, a position of distal tip 128. For example, first actuator 116 may be configured to articulate, or bend, articulation portion 105 in one or more directions (e.g., up, down, left, or right relative to a longitudinal axis of shaft 104). In other aspects, first actuator 116 may be one or more rocker switches or knobs.

Second actuator 117 may include a rocker switch, a roller, a slide potentiometer, a lever, a rotary potentiometer, or buttons configured to control a position of elevator 134 at distal tip 128. For example, second actuator 117 may adjust a position of elevator 134 up, down, or to any position in between. In some aspects, second actuator 117 may be a slider or lever configured to slide or pivot proximally or distally within a slot 120 of handle 102. For example, a proximal-most position of second actuator 117 within slot 120 may correlate to a fully closed/raised position of elevator 134, and a distal-most position of second actuator 117 may correlate to a fully open/lowered position of elevator 134, or vice versa. Any position of second actuator 117 between the fully proximal and fully distal positions may correlate to a partially open/raised or a partially closed/lowered position of elevator 134.

Third actuator 118 may be a knob, dial, wheel, a rotary potentiometer, another rotational actuator, a lever, a slider, etc. An axis of rotation of third actuator 118 may be approximately parallel to a longitudinal axis of handle 102. In aspects, third actuator 118 may function as a speed or sensitivity control for the movements or actions initiated by first actuator 116 or second actuator 117. For example, when first actuator 116 is used to control the articulation of distal tip 128, rotation of third actuator 118 may adjust the rate at which the articulation occurs or the sensitivity with which inputs from actuator 118 are transmitted. Additionally or alternatively, when second actuator 117 is used to control a position of elevator 134, rotation of third actuator 118 may adjust the rate at which the actuation occurs or the sensitivity of second actuator 117. A clockwise rotation of third actuator 118 may increase the speed of articulation/actuation, while a counterclockwise rotation may decrease the speed of articulation/actuation , or vice versa. This speed or sensitivity control may allow for both rapid positioning and fine, precise adjustments of the distal tip 128 or elevator 134 as needed during a procedure.

In some aspects, the functionality of third actuator 118 may be context-sensitive, automatically adjusting its control parameters based on which other actuators are currently in use or based on the current position of medical device 100 within the subject. For example, quick movements of articulation portion 105 or elevator 134 may be inhibited when distal tip 128 is in a smaller body lumen of the subject. In such a way, perforations of distal tip 128 through a wall of the lumen or damage to the tissue surrounding distal tip 128 may be minimized.

In some aspects, third actuator 118 may offer discrete speed settings (e.g., low, medium, high) or may provide continuous variable speed control within a defined range. The actuator may include tactile feedback mechanisms, such as detents or variable resistance, to help the user gauge the current speed setting without needing to look away from the procedure.

In other aspects, third actuator 118 may be programmed to control multiple speed-related parameters simultaneously. For instance, third actuator 118 may be configured to adjust both the articulation speed of distal tip 128 and the movement speed of elevator 134 proportionally, maintaining a consistent feel across different functions of the device.

In some aspects, the automatic adjustment of third actuator 118 may be accomplished via one or more sensors or a force feedback loop. For example, during use, articulation portion 105 may be articulated and hit, or abut against, surrounding tissue. When articulation portion 105 or distal tip 128 does hit the tissue, a resulting force may be detected by the system (e.g., via one or more sensors or via a force feedback loop) and parameters of the articulation of articulation portion 105 may be automatically adjusted, such as the speed of articulation portion 105 or the amount articulation portion 105 can be articulated. For example, when articulation portion 105 or distal tip 128 hits, or abuts against, surrounding tissue, a speed at which articulation portion 105 articulates or an amount that articulation portion 105 is capable of articulating may decrease. The automatic adjustment of articulation portion 105 may assist in preventing or limiting damage to the surrounding tissue.

Additionally or alternatively, the automatic adjustment of third actuator 118 may be used to control parameters of elevator 134. For example, as previously described, elevator 134 may be configured to bend a tool or instrument extending through shaft 104. Accordingly, elevator 134 may apply – and therefore be subjected to – increased forces, particularly when bending the tool or instrument. The third actuator 118 may therefore be used to increase the forces being applied to bend the tool or instrument, making it easier for the user to articulate elevator 134 when additional force is required. In other aspects, for example, when a fragile or larger tool or instrument is used, third actuator 118 may be used to limit the movement of elevator 134 to prevent damage to the tool or instrument while still allowing controlled articulation. The force feedback loop may incorporate one or more force sensors positioned along the articulation portion 105 or distal tip 128, including elevator 134. These sensors may be strain gauges, piezoelectric sensors, or other force-sensitive elements capable of detecting and measuring the forces exerted on the articulation portion 105 or distal tip 128 as it interacts with surrounding tissue.

The sensors may continuously monitor the forces experienced by the articulation portion 105 or distal tip 128 during movement and articulation. When the articulation portion 105 or distal tip 128 encounters resistance from tissue, the force sensors may detect an increase in applied force. This force data may be transmitted back to the handle 102, where it may be processed by a processor disposed within the handle. Additionally or alternatively, the force data may be transmitted through umbilicus to an external processor. The processor may analyze the force data in real-time, comparing it to predetermined thresholds or using algorithms to interpret the nature and magnitude of the tissue interaction. Based on this analysis, the system may automatically adjust various parameters of articulation portion 105 or control of elevator 134.

The force feedback loop may also provide haptic feedback to the user through first actuator 116 or second actuator 117. For example, when significant resistance is encountered, the system may increase the resistance felt by the user when manipulating the actuators, providing a tactile indication of the forces being experienced at the distal end of the device. The tactile indication may include a vibration, indicating to the user that a force threshold was exceeded, such as when distal tip 128 interacts with the surrounding tissue or when elevator 134 bends a tool or instrument. In other aspects, the feedback may be auditory or visual. For example, a sound may be played or a notification may be displayed on an external display (e.g., a monitor) or an integrated display (e.g., a display 124), providing an indication to the user as to how much force was or is being applied to the tissue or instrument. Although display 124 is depicted on handle 102, display 124 may be omitted.

Referring still to FIG. 1, medical device 100 may include an extension 122 extending laterally from handle 102. For example, a central axis of extension 122 may form an approximately 90-degree angle with a central axis of handle 102. Display 124 may be disposed on a surface 126 of extension 122. Surface 126 may be angled upwards, towards a user.

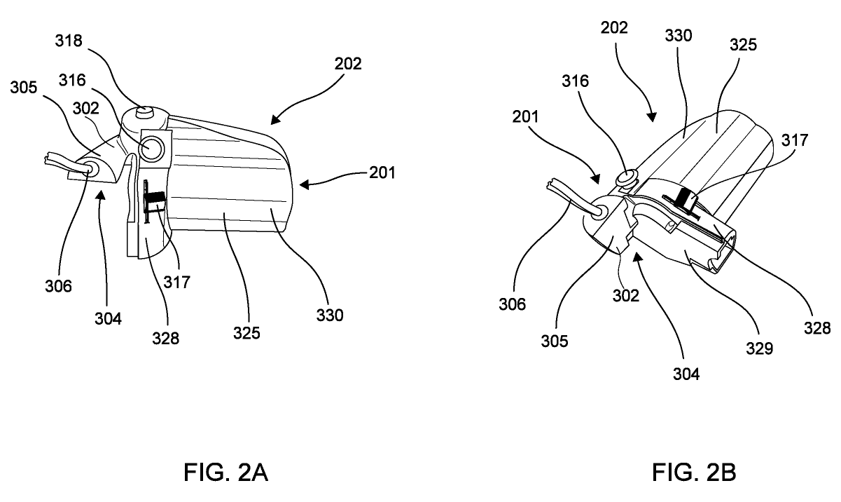

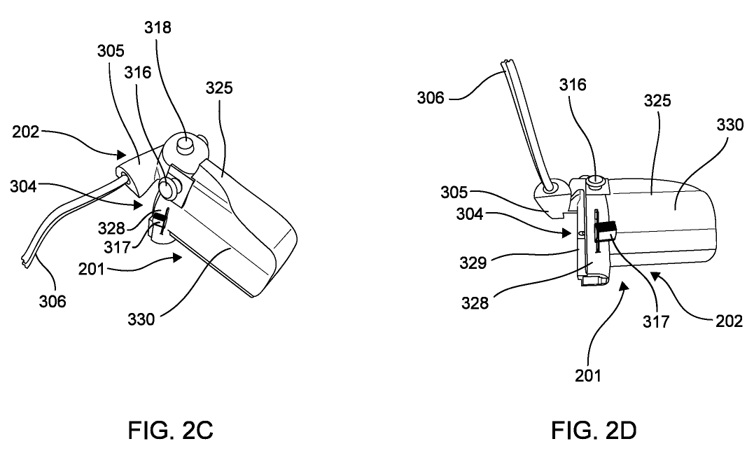

FIGS. 2A-2D depict various views of a first portion 201 of a handle 202 of a medical device, and FIGS. 3A-3D depict various views of a second portion 203 of handle 202. Together, first portion 201 and second portion 203 may be assembled to form handle 202, which may have the same or similar shape or function as handle 102 of medical device 100, shown in FIG. 1. For example, first portion 201 and second portion 203 may be combined to form handle 202, and handle 202 may be used in conjunction with shaft 104 of medical device 100. In some aspects, one or both of first portion 201 or second portion 203 may be reusable (e.g., non-disposable) or disposable. For example, first portion 201 may be a reusable (e.g., non-disposable) portion that may be configured to be used multiple times across multiple procedures. First portion 201 may be sterilizable. Second portion 203 may be a single-use or disposable portion that may be removably coupled to first portion 201.

FIGS. 2A-2D and 3A-3D may be referred to throughout the following description of handle 202. First portion 201 of handle 202 may include a housing 302. Housing 302 may define a cavity 304 for receiving and mating with a housing 211 of second portion 203. In some aspects, housing 211 may include one or more protrusions or tabs that correspond to complementary recesses or slots within cavity 304, allowing for a snap-fit or press-fit connection between the two portions. For example, the protrusions of housing 211 and recesses of cavity 304 may align and engage to secure first portion 201 and second portion 203 together. First portion 201 and second portion 203 may be secured together via various other means for removably coupling two components together. For example, first portion 201 and second portion 203 may be secured via one or more fasteners (e.g., screws, bolts, magnets, locks, etc.)

In other aspects, cavity 304 or other portions of housing 302 may feature a series of internal grooves or tracks that guide and receive corresponding ridges or rails on housing 211, allowing for a sliding connection between first portion 201 and second portion 203. Additionally or alternatively, housing 211 may include magnetic elements that align with matching magnetic elements within cavity 304 or other portions of housing 302. These magnetic elements may provide a securing force when the two portions are brought together, while still allowing for easy separation when needed.

Additionally or alternatively, cavity 304 or other portions of housing 302may incorporate a locking mechanism, such as a latch or spring-loaded pin, that engages with a corresponding feature on housing 211. This mechanism may provide audible or tactile feedback to the user when proper coupling is achieved. Cavity 304 may include flexible or compressible elements that conform to the shape of housing 211 when inserted, creating a snug fit between the two components and potentially providing environmental sealing between the two portions. The coupling mechanism may also incorporate alignment features, such as asymmetrical shapes or keyed elements, to ensure that housing 211 can only be inserted into cavity 304 in the correct orientation, preventing improper assembly.

First portion 201 may include an arm 305 extending laterally outward from a central portion 328, or a first piece of housing 302, defined between arm 305 and an extension 325. Arm 305 may extend away from a first side of central portion 328, and extension 325 may extend away from a second side of central portion 328. Extension 325 may have any of the characteristics or properties of extension 122 of FIG. 1. One or more electrical wires or cables may form a first umbilicus 306 extending from arm 305. The one or more electrical wires or cables may be electrically coupled to one or more actuators 316, 318, 317 of first portion 201 at a first end. A second end of the one or more electrical wires or cables may be electrically coupled to an external power supply, controller, monitor, etc. by, for example, a connector plug. First umbilicus 306 may lack fluidics tubing associated with, for example, air, water, or suction.

Actuators 316, 317, 318 may have any or all of the characteristics of actuators 116, 117, 118, respectively, of FIG. 1. For example, actuator 316 may be a joystick (or any other type of actuator described above) configured to control to control articulation of articulation portion 105 (FIG. 1) in multiple directions (e.g., up/down and left/right). Actuator 316 may be adjacent to arm 305 from which first umbilicus 306 extends. In aspects, actuator 316 may be disposed on central portion 328 of housing 302.

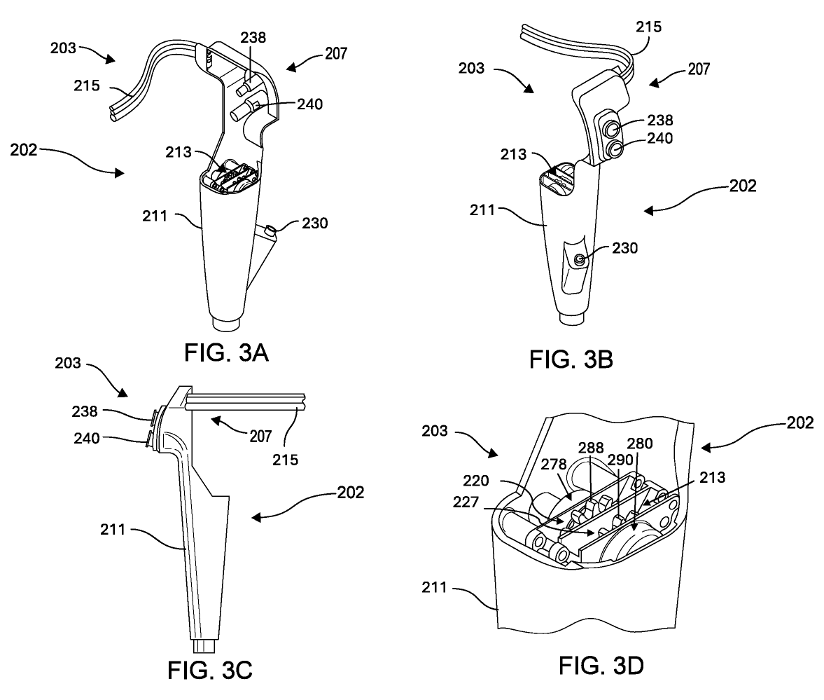

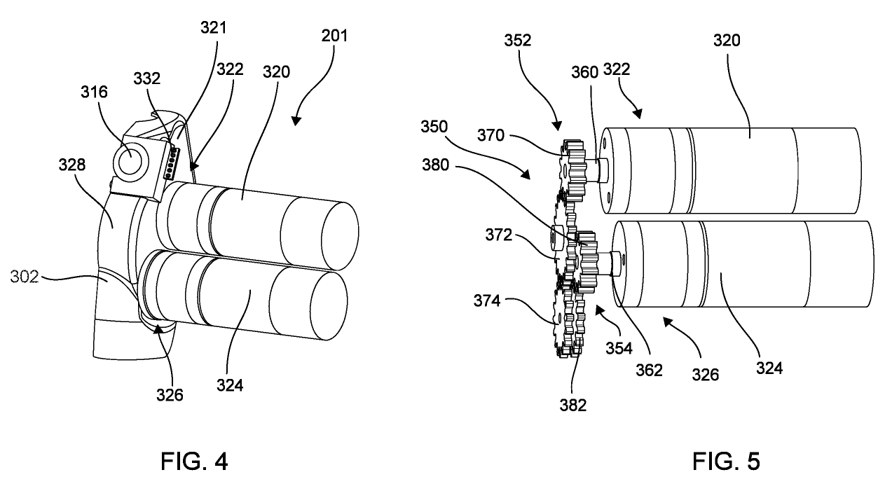

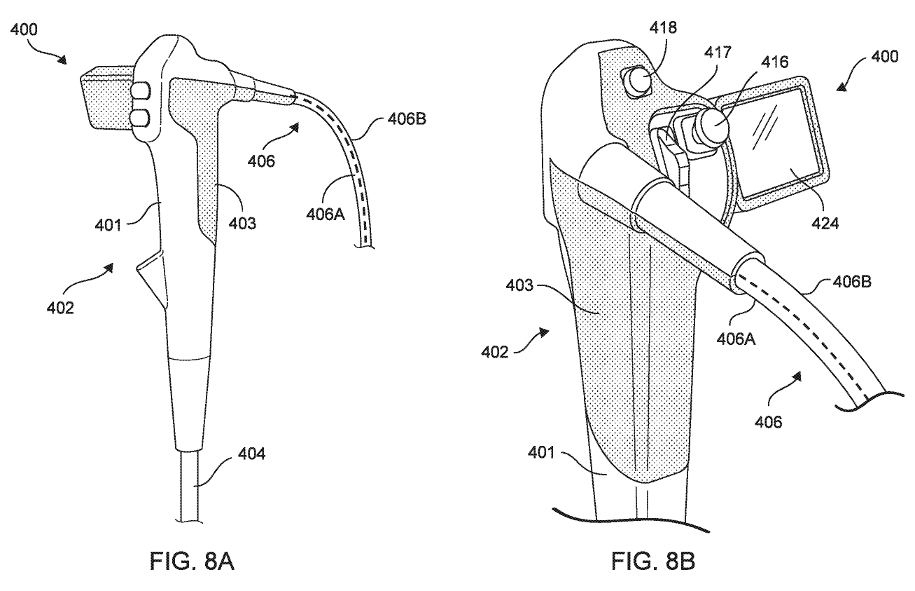

In some aspects, extension 325 may include a cover 330, or a second piece of handle housing 302 (FIGS. 2A-2D). Although not shown in FIGS. 2A-2D, a display (e.g., display 124) may be disposed on a surface of cover 330. In aspects, reusable portion 201 may include a display having any of the features of the display discussed below with respect to FIGS. 8A and 8B. Cover 330 is removed to show internal aspects of first portion 201 in FIGS. 4 and 5.

Referring primarily to FIGS. 3A-3D, second portion 203 may include housing 211. Second portion 203 may include a port 230 in communication with the working channel of shaft 104 (of FIG. 1). Second portion 203 may include a proximal extension 207. One or more fluidics tubes may together form a second umbilicus 215. Second umbilicus 215 may extend laterally away from proximal extension 207. Second umbilicus 215 may only include tubes, conduits, or other components necessary for supplying fluid (e.g., liquid or gas) or suction to medical device 100. For example, second umbilicus 215 may not include any wires or cables for communication with electrical components. In aspects, second umbilicus may include coupling devices (e.g., clips, tethers, wraps, etc.) such that second umbilicus may be secured to first umbilicus 306 of first portion 201. Accordingly, securing first umbilicus 306 with second umbilicus 215 may create a larger, single umbilicus to assist with cable and tube management during use. In alternatives, second umbilicus 215 may include such wires or cables.

Second portion 203 may include a first valve well 238 for receiving a first valve body (e.g., a suction valve) and a second valve well 240 for receiving a second valve body (e.g., an air/water valve). Second umbilicus 215 may connect medical device 100 to a source of fluid and a source of suction, and the valve bodies inserted into first and second valve wells 238, 240 may be used to control the delivery of fluids through tubing extending through shaft 206 and the application of suction through the working channel(s), respectively. Although not shown for clarity purposes, fluidics tube(s) of second umbilicus 215 may extend to one or more of valve wells 238, 340. Additional fluidics tube(s) may extend from valve wells 238, 240, distally through housing 211 and to or through shaft 104 of FIG. 1. For example, the additional fluidics tube(s) may extend distally through housing 211, on one side of a pulley wire mechanism 213. Accordingly, fluids may be applied or removed via distal tip 128 (FIG. 1).

Second portion 203 may include pull wire mechanism 213. Pull wire mechanism 213 may include a first assembly portion 220 and a second assembly portion 227. First assembly portion 220 may include a first spool 278 coupled to a first gear 288. Second assembly portion 227 may include a second spool 280 that is coupled to a second gear 290. In aspects, first spool 278 may be rotationally fixed to first gear 288, such that rotation of first gear 288 causes a corresponding rotation of first spool 278. Similarly, second spool 280 may be rotationally fixed to second gear 290, such that rotation of second gear 290 causes corresponding rotation of second spool 280.

In other words, an entirety of first assembly portion 220 may be rotatable together, and an entirety of second assembly portion 227 may be rotatable together. Additionally, first assembly portion 220 may be rotatable independent of second assembly portion 227 and vice versa. Rotation of gears 288, 290 may result in rotation of spools 278, 280, respectively. Rotation of first spool 278 or second spool 280 may move wires that are coupled to respective spools 278, 280, thereby causing articulation of articulation portion 105. For example, first spool 278 may control articulation in a first plane (e.g., up/down or left/right), and second spool 280 may control articulation in a second plane (e.g., the other of up/down or left/right). U.S. Provisional Patent Application No. 63/798,761, entitled “Partially Reusable Medical Devices and Related Methods of Use,” filed on May 2, 2025, incorporated by reference herein, describes further details of such articulation.

A proximal portion of first gear 288 and second gear 290 may be at least partially exposed at a proximal end of housing 211 of second portion 203 (e.g., in an unassembled state). Accordingly, when first portion 201 is assembled with second portion 203, corresponding gears of first portion 201 (described below) may interact with teeth of respective first gear 288 and second gear 290 of second portion 203.

FIGS. 4, 5, 6, 7A, and 7B provide various alternative views of internal aspects of first portion 201. For example, FIGS. 4-7B illustrate first portion 201 with cover 330 of FIGS. 2A-2D removed. As shown in FIGS. 4-7B, first portion 201 may include a first motor 320, a second motor 324, and a gear assembly 350.

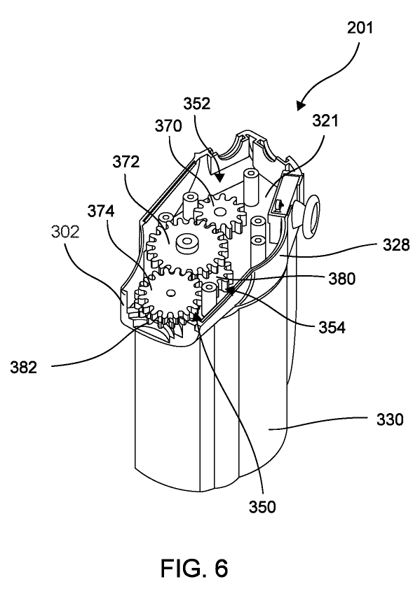

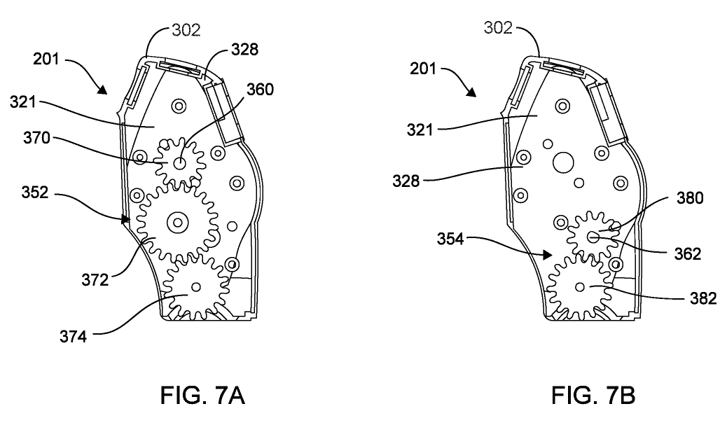

In particular, FIG. 5 shows first motor 320, second motor 324, and gear assembly 350 separate from housing 302. FIG. 6 shows gear assembly 350 within central portion 328 of housing 302. FIG. 7A shows a first gear train 352 of gear assembly 350 within central portion 328 of housing 302 to reveal features of first gear train 352. FIG. 7B shows a second gear train 354 of gear assembly 350 within central portion 328 of housing 302 to reveal features of second gear train 354.

First portion 201 may include a circuit board 332 for controlling the functions of first actuator 316 or for translating movement of first actuator 316 into movement of articulation portion 105. Circuit board 332 may be disposed within cover 330 in a fully assembled state. In aspects, circuit board 332 may be in electronic communication with first actuator 316 and with a first motor 320 and a second motor 324. Accordingly, actuation of first actuator 316 may result in the activation of first motor 320 or second motor 324 (e.g., simultaneously or individually).

First portion 201 may include first motor 320 and a second motor 324 extending laterally away from central portion 328. First motor 320 and second motor 324 may be disposed within cover 330 in a fully assembled state. A first end 322 of first motor 320 may be mounted to or otherwise adjacent to central portion 328 such that first motor 320 extends generally perpendicular to a central longitudinal axis of handle 202. Similarly, a first end 326 of second motor 324 may be mounted or otherwise adjacent to central portion 328 such that second motor 324 extends generally perpendicular to the central longitudinal axis of handle 202.

In aspects, as shown, first motor 320 may be proximal of second motor 324. In some aspects, a line extending along a central longitudinal axis of handle 202 may also extend through centers of first motor 320 and second motor 324. Actuator 316 may be proximal of second motor 324 and proximal of at least a portion of first motor 320.

First end 322 of first motor 320 may be coupled to a first axle 360 (FIGS. 5 and 7A) that extends through a wall 321 at least partially defining central portion 328. Wall 321 may separate first motor 320 and second motor 324 from gear assembly 350. First end 326 of second motor 324 may include a second axle 362 (FIGS. 5 and 7B) that extends through an opening of central portion 328. Each of first axle 360 and second axle 362 may be configured to rotate one or more gears of a gear assembly 350. For example, first motor 320 may be operative to rotate first axle 360 about a longitudinal axis of first axle 360, and second motor 324 may be operative to rotate second axle 362 about a longitudinal axis of second axle 362. The axes of first axle 360 and second axle 362 may be approximately parallel to the axes of first motor 320 and second motor 324, respectively.

First axle 360 may be fixedly coupled to a first gear 370 of first gear train 352 of gear assembly 350. Accordingly, activation of first motor 320 results in the rotation of first axle 360 and, therefore, rotation of first gear 370. Each of the gears described herein, including first gear 370, may be a round gear having a plurality of teeth defined on an outer circumference. The plurality of teeth of each gear may be configured to interact or mesh with adjacent gears, such that rotation of one gear results in the rotation of an adjacent gear.

For example, the teeth of first gear 370 may mesh with the teeth of a second gear 372, and teeth of second gear 372 may mesh with the teeth of third gear 374. First gear train 352 may include second gear 372 and third gear 374. Accordingly, when first gear 370 is rotated in a first direction, second gear 372 is rotated in a second, opposite direction, and third gear 374 is rotated in the first direction simultaneously. Second gear 372 may be disposed between first gear 370 and third gear 374. For example, first gear 370 may be a proximalmost gear, and third gear 374 may be a distalmost gear. Second gear 372 may be larger in diameter than third gear 374 and first gear 370. Second gear 372 may be an idler gear, in some examples. Third gear 374 may be larger in diameter than first gear 370. First gear train 352 may include additional gears (e.g., more than three gears) or fewer gears (e.g., fewer than three gears).

Similarly, second axle 362 may be fixed to a fourth gear 380 of second gear train 354. Second gear train 354 may further include a fifth gear 382. For example, teeth of fourth gear 380 may mesh with teeth of a fifth gear 382. Accordingly, activation of second motor 324 may result in the rotation of second axle 362 and, therefore, rotation of fourth gear 380 in a first direction and fifth gear 382 in a second, opposite direction.

Fourth gear 380 may be a proximalmost gear of second gear train 354, and fifth gear 382 may be a distalmost gear of second gear train 354. Fifth gear 382 may be larger in diameter than fourth gear 380. In some examples, fifth gear 382 may have a same size (e.g., diameter) as fourth gear 380. In some examples, first gear 370 may have approximately the same diameter as fourth gear 380, and third gear 374 may have approximately the same diameter as fifth gear 382. Thus, first gear train 352 may have a same gear ratio as second gear train 354.

Second gear train 354 may include additional gears (e.g., more than two gears) or only one gear. In some aspects, second gear train 354 may include fewer gears as compared to first gear train 352. The different number of gears may be due to the fact that first motor 320 is proximal of second motor 324. As shown in FIG. 6, third gear 374 of first gear train 352 and fifth gear 382 of second gear train 354 may be approximately longitudinally aligned with one another and may, in some aspects, share a common axis of rotation. A size of second gear 372 may be chosen to align third gear 372 and fifth ger 382 longitudinally. This may facilitate coupling of first portion 201 to second portion 203, as described below.

A cover 329 (FIG. 2D) may be coupled to central portion 328 to enclose first gear train 352 and second gear train 354 on at least one side. A distal end of central portion 328 may remain open such that the gears of first portion 201 may interact with the gears of second portion 203, as described below. Cover 329 is removed in FIGS. 6, 7A and 7B to illustrate the configuration of first gear train 352 and second gear train 354 within central portion 328.

As explained in U.S. Provisional Patent Application No. 63/798,761, entitled “Partially Reusable Medical Devices and Related Methods of Use,” filed on May 2, 2025, and incorporated by reference herein, sizes of gears first gear 370, second gear 372, and third gear 374, and fourth gear 380 and fifth gear 382, may be chosen to impart a desired mechanical advantage and speed of articulation. The gears 370, 372, 374, 380, and 382 may also have any of the properties of the gears disclosed in U.S. Provisional Patent Application No. 63/798,761, entitled “Partially Reusable Medical Devices and Related Methods of Use.” For example, some of gears 370, 372, 374, 380, and 382 may be compound gears. Furthermore, the number and size of gears 370, 372, 374, 380, and 382 are merely exemplary.

Third gear 374 of first gear train 352 and fifth gear 382 of second gear train 354 may interact with gears of second portion 203 to steer articulation portion 105. For example, the teeth of third gear 374 and the teeth of fifth gear 382 may be configured to mesh with teeth of respective first gear 288 and second gear 290 of second handle portion 203. Accordingly, when first portion 201 is assembled with second portion 203, rotation of the gears of first gear train 352 and second gear train 354 of first portion 201 results in the rotation of gears 288, 290 of second handle portion 203. For example, one of first gear train 352 and second gear train 354 may control movement of articulation portion 105 (FIG. 1) of medical device 100 along a first plane (e.g., up/down or left/right), and the other of first gear train 352 and second gear train 354 may control movement of articulation portion 105 (FIG. 1) of medical device 100 along a second plane (e.g., the other of up/down or left/right).

In aspects, gears 370, 372, 374, 380, and 382 may be configured such that articulation portion 105 articulates with the same speed in the up/down and left/right directions. Additionally or alternatively, first motor 320 and second motor 324 may be calibrated to provide the same articulation speed in different planes. For example, first gear train 352 and second gear train 354 may have the same gear ratio.

In some aspects, medical device 100 may include an indicator system to confirm that first portion 201 and second portion 203 are adequately coupled. This indicator system may provide visual, auditory, or tactile feedback to the user, ensuring proper assembly of the device. For example, the indicator may be displayed on display 124 and/or another external display (e.g. a monitor). Additionally or alternatively, the indicator may be a light having multiple colors to indicate the coupling status of first portion 201 with second portion 203. For example, a green light may signaling to the user that the device is ready for use. In contrast, if the coupling is incomplete or incorrect, solid or flashing red or yellow light may signal the user to check the connection.

The indicator system may incorporate tactile elements and/or audible signals additionally or alternatively. For instance, a small vibration or pulse may be felt through handle 102 when first portion 201 and second portion 203 are successfully coupled. Additionally or alternatively, a distinct click or beep may be emitted when the two portions are properly aligned and secured.

In some cases, the indicator system may be integrated with the device's electronic systems. A sensor within cavity 304 may detect the presence and proper alignment of housing 211. This sensor may communicate with a microprocessor in first portion 201, which may then activate the appropriate indicator (visual, auditory, and/or tactile) to confirm proper coupling.

Aspects of handle 202 may provide for customizability for a user, as well as cost savings versus entirely disposable scopes by allowing first portion 201 to be reused among different procedures. For example, handle 202 may be part of a modular system. First portion 201 may be customized for a variety of users. For example, a size or arrangement of first portion 201, including the size or placement of each actuator, may be customizable. Actuator 316 may also be customizable in, for example, its sensitivity or feedback. The same first portion 201 may be used with second portions 203 that are coupled to different types of shafts. For example, the same first portion 201 may be used with second portions 203 coupled to forward-facing endoscope shafts, side-facing duodenoscope shafts, or smaller cholangioscope shafts. The same second portion 203 may also be used with multiple first portions 201. For example, the same second portion 203 may be used with first portion 201 disclosed herein or the reusable portions disclosed in U.S. Provisional Patent Application No. 63/798,761, entitled "Partially Reusable Medical Devices and Related Methods of Use.”

FIGS. 8A and 8B depict an alternative exemplary medical device 400. Medical device 400 may have any of the properties of medical device 100 or of the medical devices or aspects thereof disclosed in U.S. Provisional Patent Application No. 63/798,639, entitled “Handle Systems and Assemblies for Medical Devices and Related Methods of Use.” For example, medical device 400 may include a handle 402 comprised of a first portion 401 and a second portion 403. First portion 401 and second portion 403 may be removably coupled together. In aspects, one or both of first portion 401 or second portion 403 may be reusable (e.g., non-disposable) or disposable. First portion 401 and second portion 403 may have the same characteristics as first portion 201 and second portion 203, described above with respect to FIGS. 2A-7B, except as described below.

Medical device 400 may further include an umbilicus 406 extending from a proximal portion of a handle 402. Shown in broken lines, umbilicus 406 may be comprised of a first umbilicus 406A (e.g., similar to umbilicus 306 of FIGS. 2A-2D) and of a second umbilicus (e.g., similar to umbilicus 215 of FIGS. 3A-3C). For example, first portion 401 may include first umbilicus 406A, and second portion 403 may include second umbilicus 406B.

A shaft 404 may extend distally from a distal end of handle 402. Although not shown, a distal end of shaft 404 may include an articulation portion and a distal tip including an imaging device, an illumination device, and an elevator (e.g., see articulation portion 105, distal tip 128, imaging device 130, illumination device 132, and elevator 134 of medical device 100, shown in FIG. 1).

Medical device 400 may further include a plurality of actuators, including a first actuator 416, a second actuator 417, or a third actuator 418, each configured to control various functions of medical device 400. Although three actuators are shown, medical device 400 may include fewer actuators or additional actuators. In aspects, first actuator 416, second actuator 417, and third actuator 418 may have any or all of the characteristics of first actuator 116, second actuator 117, or third actuator 118, discussed above with respect to FIGS. 1, 2A-2D.

In some aspects, first actuator 416 may be a joystick configured to control a position or configuration of the articulation portion, and, therefore, a position of the distal tip of medical device 400. Second actuator 417 may include a rocker switch or a roller configured to control a position of the elevator of medical device 400.

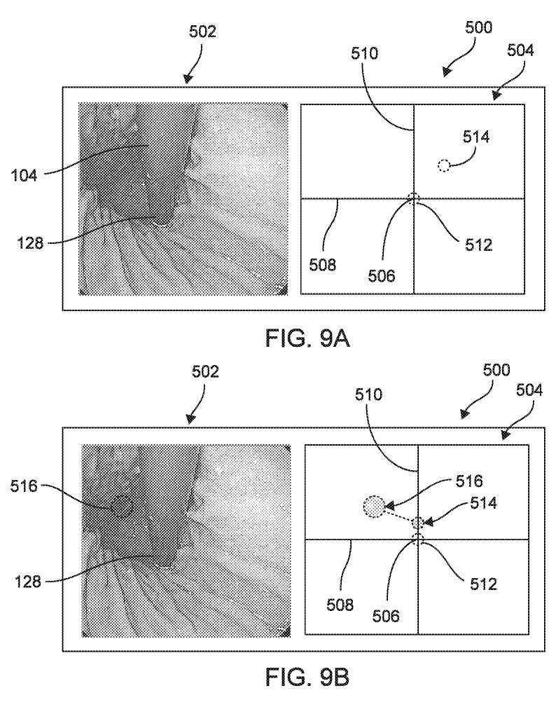

Third actuator 418 may be a knob, dial, wheel, a rotary potentiometer, another rotational actuator, a lever, a slider, etc. In other aspects, third actuator 418 may be a button or a switch. Third actuator 418 may be configured to adjust the rate at which articulation occurs or the sensitivity with which inputs first actuator 416, second actuator 417, or third actuator 418 are transmitted. Additionally or alternatively, third actuator 418 may be configured to control aspects of a graphical user interface (GUI) 500, described in further detail below with respect to FIGS. 9A and 9B.

Medical device 400 may further include an integrated display 424. Integrated display 424 may have any or all of the characteristics of display 124, described above. In some aspects, integrated display 424 may be a touchscreen display controlled by a user’s finger, or may be controlled by one or more actuators, such as actuator 418. For example, actuator 418 may be configured to record a video or image, control a brightness of integrated display 424 or of illumination device 132, or control other aspects of integrated display 424.

FIGS. 9A and 9B illustrate an exemplary graphical user interface (GUI) 500 configured to be displayed on integrated display 424 or an external or other display (such as a monitor). For example, FIG. 9A illustrates a first view of GUI 500 where a target is not identified, and FIG. 9B illustrates a second view of GUI 500 where a target is identified using an identifier, discussed in further detail below. In aspects, all or portions of GUI 500 may be displayed on integrated display 424 or the external display. For example, GUI 500 may include a first graphical user interface portion or display 502 and a second graphical user interface portion or display 504.

First display 502 may be configured to provide a live image feed of imaging device 130, and second display 504 may be configured to provide a live feed of a schematic of a location of the distal tip (such as distal tip 128 of FIG. 1) relative to a nominal position.

Second display 504 may be divided by four quadrants, separated by an X-axis 508 and a Y-axis 510. In some aspects, the nominal position may be when the distal tip is aligned with the longitudinal axis of shaft 104 in a straight configuration. In other aspects, the nominal position may be customized by the user. For example, the nominal position may be when the distal tip is at an angle of approximately 45 degrees relative to the longitudinal axis of the shaft in a straight configuration. Accordingly, the nominal position may be set and re-set once or multiple times throughout a procedure. The nominal position may assist a user in identifying a starting position or a desired position of the distal tip.

In some aspects, the nominal position may correlate to an origin 506 on second display 504. For example, origin 506 may be the point at which X-axis 508 and Y-Axis 510 intersect. A first identifier 512 may assist a user in identifying the nominal position. First identifier 512 may include any shape (such as a circle, as shown, a square, a rectangle, a triangle, a polygon, or any other shape) having any size and color. In aspects, the size, shape, and color of first identifier 512 may be customized by the user.

Second display 504 may further include a second identifier 514 configured to assist a user in identifying a position of the distal tip relative to the nominal position. For example, when the nominal position is set when distal tip is aligned with the longitudinal axis of the shaft in a straight configuration, when the distal tip is articulated up, down, left, or right, second identifier 514 may move accordingly relative to origin 506. Second identifier 514 may include any shape (such as a circle, as shown, a square, a rectangle, a triangle, a polygon, or any other shape) having any size and color. In aspects, the size, shape, and color of second identifier 514 may be customized by the user.

Shown in FIG. 9B, first display 502 and second display 504 may each include a third identifier 516 configured to assist a user in identifying a position of a target during a procedure. For example, third identifier 516 may be an overlay on first display 502 to show a position of a target during the procedure. The position of third identifier 516 may on first display 502 may correlate with the position of third identifier 516 on second display 504, for example. Third identifier 516 may assist a user in identifying a position of the distal tip relative to the target. For example, when the distal tip is aligned with the target, second identifier 514 and third identifier 516 may overlap. Similarly, when the target is aligned with the nominal position of the distal tip, first identifier 512 and third identifier 516 may overlap. All three identifiers 512, 514, 516 may overlap, for example, when the distal tip is in the nominal position and the target is directly in front of the distal tip. In some aspects, first display 502 and second display 504 may include multiple third identifiers 516, for example, when multiple targets are identified in a live image. The user may turn on or off one or more identifiers 512, 514, 516. For example, the user may choose to show none, all, or a desired selection of first identifier 512, second identifier 514, or third identifier 516 on one or both of first display 502 and second display 504.

Third identifier 516 on one or both of first display 502 and second display 504 may include any shape (such as a circle, as shown, a square, a rectangle, a triangle, a polygon, or any other shape) having any size and color. In aspects, the size, shape, and color of third identifier 516 may be customized by the user.

In some aspects, one of identifiers 514 or 516 may be an identifier of a location tagged by a user. For example, a user may navigate the distal tip to a desired location and then may activate an actuator (e.g., actuator 418) or use a touch-screen interface to mark the location with an identifier 514 or 516. The user may navigate to a different location and may use the identifier 514 or 516 to return the distal tip to the previous position.

It will be apparent to those skilled in the art at various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

What is claimed is:

1. A medical device comprising:
a first portion comprising:
 a housing;
an actuator;
a motor configured to be activated by the actuator;
a gear assembly coupled to the motor; and
a second portion removably coupled to the first portion, the second portion comprising a spool coupled to a wire and a second gear coupled to the spool;
wherein a first gear of the gear assembly is configured to interact with the second gear to move the wire.

2. The medical device of claim 1, wherein the second gear of the spool extends proximally relative to a housing of the second portion.

3. The medical device of claim 1, wherein the motor is a first motor, the gear assembly is a first gear assembly, the spool is a first spool, and the wire is a first wire, wherein:
the first portion further comprises:
a second motor configured to be activated by the actuator; and
a second gear assembly coupled to the second motor; and
the second portion further comprises:
a second spool coupled to a second wire and a fourth gear coupled to the second spool;
wherein a third gear of the second gear assembly is configured to interact with the fourth gear to move the second wire.

4. The medical device of claim 3, wherein each of the first motor and the second motor extends approximately perpendicularly to a central longitudinal axis of the medical device.

5. The medical device of claim 3, wherein the first motor is coupled to a first axle that rotates a fourth gear of the first gear assembly, and wherein the second motor is coupled to a second axle that rotates a fifth gear of the second gear assembly, wherein the first axle and the second axle are approximately perpendicular to a central longitudinal axis of the medical device.

6. The medical device of claim 3, wherein the medical device further includes a shaft extending from the second portion, wherein a distal end of the shaft includes an articulation portion, and wherein movement of the first wire articulates the distal end of the shaft in a first direction and a second direction, opposite the first direction.

7. The medical device of claim 6, wherein movement of the second wire articulates the distal end of the shaft in a third direction and a fourth direction, wherein the fourth direction is opposite the third direction, and wherein the third direction and the fourth direction are perpendicular to the first direction and the second direction.

8. The medical device of claim 3, wherein the second gear assembly includes more gears than the first gear assembly.

9. The medical device of claim 8, wherein the first gear assembly includes three gears and the second gear assembly includes two gears.

10. The medical device of claim 3, wherein the first motor is proximal of the second motor.

11. The medical device of claim 3, wherein the first gear assembly is coupled to the first motor via a first axle, wherein the second gear assembly is coupled to the second motor via a second axle, wherein each of the first axle and the second axle extends through a wall separating the first motor and the second motor from the first gear assembly and the second gear assembly, respectively.

12. The medical device of claim 1, wherein the first portion includes a first umbilicus, wherein the first umbilicus includes at least one electrical wire or cable.

13. The medical device of claim 12, wherein the second portion includes a second umbilicus, wherein the second umbilicus includes at least one fluidics tube.

14. The medical device of claim 13, wherein the second portion includes a first valve well configured to receive a valve body for controlling application of a fluid.

15. The medical device of claim 1, wherein the actuator is a first actuator, wherein the medical device further includes:
a shaft extending from a distal end of the second portion, wherein a distal portion of the shaft includes an articulation portion and an elevator; and
a second actuator disposed on the first portion, wherein the first actuator is configured to articulate the articulation portion of the shaft in at least one direction, and wherein the second actuator is configured to actuate the elevator.

16. A medical device comprising:
a handle having:
a first portion including:
a motor; and
a first gear, wherein the motor is configured to rotate the first gear; and
a second portion removably coupled to the first portion, wherein the second portion includes:
a second gear configured to interact with the first gear of the first portion to rotate a spool; and
a wire coupled to the spool; and
a shaft extending from the second portion, wherein a distal end of the shaft includes an articulation portion, wherein the wire is coupled to the articulation portion such that, upon activation of the motor, the wire articulates the articulation portion in a first direction or a second direction, opposite the first direction.

17. The medical device of claim 16, wherein the motor is a first motor, the gear is a first gear, and the wire is a first wire:
wherein the first portion further comprises:
a second motor; and
a third gear,
wherein the second portion further comprises:
a fourth gear configured to interact with the third gear of the first portion to rotate a second spool; and
a second wire coupled to the second spool and to the articulation portion,
wherein, upon activation of the second motor, the second wire articulates the articulation portion of the shaft in a third direction or a fourth direction.

18. The medical device of claim 17, wherein the first gear is a gear of a first gear train, wherein the third gear is a gear of a second gear train, wherein the first gear train includes more gears than the second gear train.

19. A medical device, comprising:
a first handle portion, the first handle portion including:
an actuator;
a first motor coupled to a first gear via a first axle, wherein the first motor is configured to rotate the first gear;
a second motor coupled to a second gear via a second axle, wherein the second motor is configured to rotate the second gear; and
a second handle portion, wherein the second handle portion is removably coupled to the first handle portion, wherein the second handle portion includes:
a first wire connected to a first spool, wherein the first spool is configured to rotate to move the first wire proximally or distally;
a second wire connected to a second spool, wherein the second spool is configured to rotate to move the second wire proximally or distally;
wherein, upon actuation of the actuator, the first gear rotates the first spool or the second gear rotates the second spool.

20. The medical device of claim 19, wherein actuation of the actuator is configured to simultaneously activate the first motor and the second motor in order to simultaneously rotate the first spool and the second spool.
